(19) United States
(12) Patent Application Publication
(10) Pub. No.: US 2026/0198780 A1
(43) Pub. Date: Jul. 16, 2026

(54) MRI BRAIN IMAGE-BASED SYSTEM FOR RAPID DIFFERENTIATION OF NORMAL PRESSURE HYDROCEPHALUS, ALZHEIMER'S DISEASE, AND NORMAL CONDITION

(71) Applicants: KYUNGPOOK NATIONAL UNIVERSITY INDUSTRY-ACADEMIC COOPERATION FOUNDATION, Daegu (KR); DAEGU CATHOLIC UNIVERSITY INDUSTRY ACADEMIC COOPERATION FOUNDATION, Gyeongsangbuk-do (KR); AICU Corp., Daegu (KR)

(72) Inventors: KYUNG HUN KANG, Daegu (KR); UI CHEUL YOON, Daegu (KR); HO SANG YU, Daegu (KR); EUN AH HWANG, Daegu (KR)

(21) Appl. No.: 19/175,104

(22) Filed: Apr. 10, 2025

(30) Foreign Application Priority Data
Jan. 16, 2025 (KR) 10-2025-0006842
Jan. 24, 2025 (KR) 10-2025-0011122

(51) Int. Cl. A61B 5/00; A61B 5/055; G06T 7/00

(52) U.S. Cl. CPC A61B 5/0042; A61B 5/055; A61B 5/4088; A61B 5/7267; G06T 7/0012; A61B 2576/026; G06T 2207/10088; G06T 2207/20021; G06T 2207/20081; G06T 2207/30016

(57) ABSTRACT

A method for assessing a patient's brain disease state from brain images includes acquiring the brain images, partitioning them into predefined regions based on anatomical landmarks, extracting disease-indicative features, using a pretrained model to generate a disease-associated biomarker from the features, and determining the patient's brain disease state based on the biomarker.

### CROSS REFERENCE TO RELATED APPLICATIONS AND CLAIM OF PRIORITY

This application claims the benefit under 35 USC §119 of Korean Patent Application Nos. 10-2025-0006842 filed on January 16, 2025 and 10-2025-0011122 filed on January 24, 2025 in the Korean Intellectual Property Office. The disclosures of the priority applications are hereby incorporated by reference in their entireties.

### BACKGROUND

### 1. Technical Field

The present disclosure relates to a method and apparatus for assessing a brain disease in a patient, and more particularly, to a method and apparatus for assessing a brain disease based on brain imaging.

### 2. Technical Considerations

Deep learning involves training on extremely large datasets to probabilistically select the most likely outcome when presented with new data. Due to its adaptability to various images and its capacity to automatically extract features during model training based on data, deep learning is increasingly being explored for applications in artificial intelligence.

Extensive research is underway to apply deep learning technology to the medical field, aiming for rapid and accurate assessment of patient diseases.

### SUMMARY

According to various embodiments, a method and apparatus can be provided for assessing a brain disease state in a patient based on brain images, using a trained artificial intelligence model.

According to one embodiment, there may be provided a method of assessing a brain disease, the method including: acquiring a set of brain images of a patient; partitioning the brain images into a plurality of predefined regions based on anatomical landmarks; extracting feature information from the plurality of regions, wherein the feature information is indicative of a disease state; generating, based on a pre-trained brain disease assessment model, a biomarker associated with the disease from the extracted feature information, wherein the biomarker is generated in at least one of the plurality of regions; and determining a status of the brain disease in the patient based on the biomarker.

In some non-limiting embodiments or aspects, the plurality of regions includes: a tight high-convexity region of the cerebrum, a region encompassing enlarged Sylvian fissures, a region exhibiting ventriculomegaly, and an intracranial region.

In some non-limiting embodiments or aspects, the state of the disease includes normal pressure hydrocephalus or Alzheimer's disease.

In some non-limiting embodiments or aspects, the extracting of the feature information from the plurality of regions includes: extracting, from each of the plurality of regions, imaging features including at least one of volume, shape, signal intensity, and texture.

In some non-limiting embodiments or aspects, determining the status of the brain disease in the patient includes: analyzing a quantitative value of the biomarker; and comparing the quantitative value to a predetermined reference state, thereby assessing a risk of normal pressure hydrocephalus or Alzheimer's disease.

In some non-limiting embodiments or aspects, determining the status of the brain disease in the patient includes: predicting a presence and progression of the disease based on the biomarker and the feature information.

In some non-limiting embodiments or aspects, the brain disease assessment model is generated by: pre-configuring an artificial intelligence model to generate a biomarker associated with the disease in one of a plurality of regions partitioned within training brain images, based on feature information extracted from training brain images; and training the artificial intelligence model.

In some non-limiting embodiments or aspects, the method further includes, prior to pre-configuring the artificial intelligence model, performing at least one of: acquiring a dataset including brain images from a plurality of patients as the training brain images; partitioning the training brain images within the dataset into a plurality of regions; and extracting feature information for detecting the disease state from the plurality of regions of the training brain images.

In some non-limiting embodiments or aspects, the brain disease assessment model is trained to: partition the training brain images into a plurality of regions; or extract feature information for detecting the state of the disease from the plurality of regions of the training brain images.

In some non-limiting embodiments or aspects, the method may further include re-training the brain disease assessment model based on the dataset further including brain images of the patient.

According to another embodiment, there may be provided a brain disease determination apparatus, including: a memory storing a brain image processing model trained to generate a biomarker associated with the disease; and a processor configured to: acquire brain images of a patient; partition the brain images into a plurality of predefined regions based on anatomical criteria; extract feature information for detecting a state of the disease from the plurality of regions; generate, based on the brain image processing model, a biomarker associated with the disease from the feature information in at least one of the plurality of regions; and determine a status of the brain disease in the patient based on the biomarker.

In some non-limiting embodiments or aspects, the processor is configured to partition the brain images into: a tight high-convexity region of the cerebrum, a region encompassing enlarged Sylvian fissures, a region exhibiting ventriculomegaly, and an intracranial region.

In some non-limiting embodiments or aspects, the state of the disease includes normal pressure hydrocephalus or Alzheimer's disease.

In some non-limiting embodiments or aspects, the processor is configured to extract, from each of the plurality of regions, imaging features including at least one of volume, shape, signal intensity, and texture.

In some non-limiting embodiments or aspects, the processor is configured to: analyze a quantitative value of the biomarker; and compare the quantitative value to a predetermined reference state, thereby assessing a risk of normal pressure hydrocephalus or Alzheimer's disease.

In some non-limiting embodiments or aspects, the processor is configured to predict a presence and progression of the disease based on the biomarker and the feature information.

In some non-limiting embodiments or aspects, the brain disease assessment model is generated by: pre-configuring an artificial intelligence model to generate a biomarker associated with the disease in one of a plurality of regions partitioned within training brain images, based on feature information extracted from training brain images; and training the artificial intelligence model.

In some non-limiting embodiments or aspects, prior to pre-configuring the artificial intelligence model, the processor is configured to perform at least one of: acquiring a dataset including brain images from a plurality of patients as the training brain images; partitioning the training brain images within the dataset into a plurality of regions; and extracting feature information for detecting the disease state from the plurality of regions of the training brain images.

In some non-limiting embodiments or aspects, the brain disease assessment model is trained to: partition the training brain images into a plurality of regions; or extract feature information for detecting the state of the disease from the plurality of regions of the training brain images.

In some non-limiting embodiments or aspects, the processor is configured to re-train the brain disease assessment model based on the dataset further including brain images of the patient.

According to various embodiments, there is provided a method and apparatus for assessing a patient's brain disease state from brain images, thereby rapidly and accurately differentiating normal pressure hydrocephalus and Alzheimer's disease, which can reduce diagnostic time and provide patients with faster treatment planning.

According to various embodiments, there is provided a method and apparatus for assessing a patient's brain disease state from brain images, which minimizes determination errors and provides consistent results through an automated analysis process, thereby supporting healthcare professionals in making more accurate assessments.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 schematically illustrates an apparatus configuration for assessing a brain disease state from brain images, according to one embodiment.

FIG. 2 schematically illustrates a brain image processing model configuration for assessing a brain disease state in an apparatus, according to one embodiment.

FIG. 3 is a flowchart showing the operational flow of an apparatus assessing a brain disease state from brain images, according to one embodiment.

FIG. 4 is a flowchart showing the operational flow for training a brain image processing model to assess a brain disease state in an apparatus, according to one embodiment.

FIG. 5 is a flowchart showing the operational flow for re-training a brain image processing model to assess a brain disease state in an apparatus, according to one embodiment.

### DETAILED DESCRIPTION

Hereinafter, embodiments will be described in detail with reference to the accompanying drawings. However, since various changes may be made in the embodiments, the scope of the patent disclosure is not limited or restricted by these embodiments. It should be understood that all modifications, equivalents, and alternatives for the embodiments are included in the scope of the present disclosure.

It will be understood that when a component is described to as being “connected,” “combined” or “coupled” to another component, the component may be directly connected or coupled the another component, but it may be “connected,” “combined” or “coupled” to the another component intervening another component may be present.

Further, in describing the components of the embodiment, the meaning of “or” may mean each of the components, may mean two or more of the components, or may mean all of the components. For example, it should be understood that the expressions “a, b or c” represent any one of “a,” “b,” “c,” “a and b,” “a and c,” “b and c,” and “a, b and c.”

Components included in one embodiment and components including common functions will be described using the same names in other embodiments. The description given in one embodiment may be applied to other embodiments, and therefore will not be described in detail within the overlapping range, unless there is a description opposite thereto.

The device and/or ‘data’ processed by the device may be expressed in terms of ”information”. Here, the information may be used as a concept including the data.

Hereinafter, various embodiments of the present disclosure will be described with reference to the accompanying drawings. However, the drawings attached to the present specification serve to further understand the technical idea together with the detailed description, such that the present disclosure should not be construed as being limited only to the illustrations of the drawings.

This disclosure describes a method and apparatus for assessing a patient's brain disease state. More specifically, it describes a method and apparatus for assessing a brain disease state from a patient's brain images by using an artificial intelligence-based learning model.

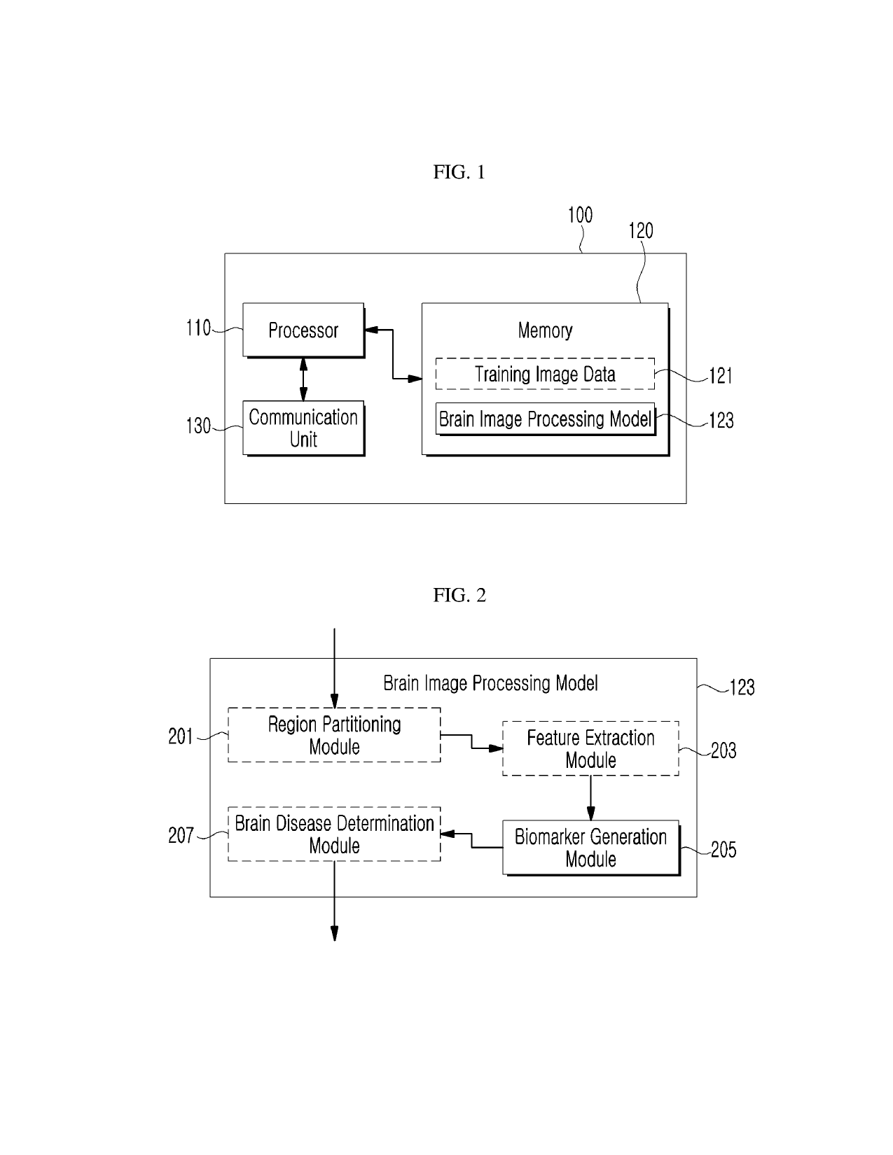

For example, according to one embodiment of the present disclosure, a method and apparatus for assessing a brain disease state can determine normal pressure hydrocephalus, which shows symptoms similar to dementia but is treatable, Alzheimer's-type dementia, which aims to slow down the progression or alleviate symptoms, or a normal state. In this regard, FIG. 1 is a diagram schematically illustrating a configuration of an apparatus for determining a brain disease state from brain images, according to one embodiment. And, FIG. 2 is a diagram schematically illustrating a configuration of a brain image processing model for determining a brain disease state in an apparatus, according to one embodiment.

Referring to FIG. 1, an apparatus 100 for determining a brain disease state from brain images (hereinafter, apparatus 100) may include a processor 110, a memory 120, and a communication unit 130.

The processor 110 includes at least one processor and may process various data for the operation of the apparatus 100 through at least one program (application, tool, plug-in, software, etc.).

The memory 120 may store various data processed by at least one component of the apparatus 100 (e.g., the processor 110 or the communication unit 130, etc.). The data may include, for example, a program for processing control commands, data processed through the program, or input data and output data related thereto.

In addition, at least one program stored in the memory 120 may include an artificial intelligence algorithm based on at least some of artificial neural network algorithms, blockchain algorithms, deep learning algorithms, regression analysis algorithms, and related mechanisms, operators, language models, and big data to perform operations of the apparatus 100.

According to one embodiment, the memory 120 may include a brain image processing model 123 configured to determine a patient's brain disease state from brain images.

The brain image processing model 123 may be configured based on at least some of various artificial intelligence learning techniques, such as Inception, MobileNet, DenseNet, Residual Network (ResNet), Recurrent Neural Network (RNN), Long Short-Term Memory (LSTM), Vision Transformer (ViT), U-Net, Random Forest, XGBoost, Scale-Invariant Feature Transform (SIFT), or Convolutional Neural Network (CNN).

Based on this, as shown in FIG. 2, the brain image processing model 123 may include at least one module based on the operations (or functions) it performs.

For example, the brain image processing model 123 may include a biomarker generation module 205 configured to generate a biomarker on a brain image based on feature information extracted for the brain image.

However, the brain image processing model 123 may further include at least some of a region partitioning module 201 that partitions the brain images into a plurality of regions (analysis target regions), a feature extraction module 203 that extracts feature information for the partitioned plurality of analysis target regions, and a brain disease determination module 207 that determines the patient's state based on the biomarker.

In addition, the memory 120 may further include training image data 121 for training the brain image processing model 123.

The training image data 121 may include brain images obtained by capturing the brains of a plurality of patients. According to one embodiment, the training image data 121 may include images obtained from various medical image databases external to the apparatus 100, such as ADNI (Alzheimer's Disease Neuroimaging Initiative), OpenNeuro, OASIS (Open Access Series of Imaging Studies), or XI dataset.

However, without being limited thereto, the training image data 121 may include brain images used in the apparatus 100 to determine the patient's brain disease state.

Herein, at least some of the training image data 121 may be configured as a dataset for training the brain image processing model 123. When the training image data 121 is configured as a dataset, the training image data 121 may be classified into at least some categories of a training dataset, a validation dataset, and a test dataset.

The communication unit 130 may support establishing a wired communication channel, establishing a wireless communication channel, and performing communication through the established communication channel, within the apparatus 100, between the apparatus 100 and at least one other device (e.g., a user device or a server), or both.

In addition, although not shown in FIG. 1, the apparatus 100 may further include at least one input/output unit.

The input/output unit may include or be connected to at least some of an input unit (not shown) that inputs data, such as a keyboard, mouse, or touchpad, and an output unit (not shown) that outputs data, such as a display, speaker, or actuator.

According to various embodiments of the present invention, the apparatus 100 or a user device connected to the apparatus 100 may include at least some of the functions of all information and communication devices, including mobile communication terminals, multimedia terminals, wired terminals, fixed terminals, and Internet Protocol (IP) terminals.

The apparatus 100, as a device for processing control commands, may be configured to include at least some functions of a workstation or a large-capacity database, or to be connected to them through communication.

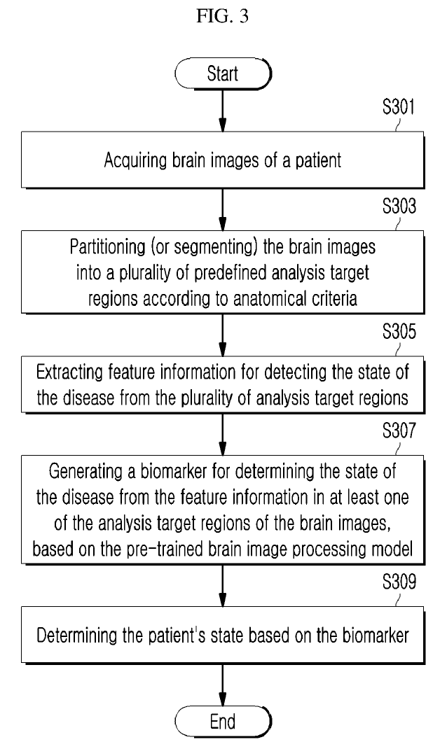

Hereinafter, a method for the apparatus 100 to assess a patient's brain disease state from brain images will be described in detail with reference to FIG. 3. In this regard, FIG. 3 is a flowchart illustrating a flow of operations of an apparatus for assessing a brain disease state from brain images according to one embodiment.

In step 301, the processor 110 may acquire brain images of a patient for whom a brain disease state is to be determined (or assessed).

According to one embodiment, the brain images acquired by the processor 110 may include images captured based on Magnetic Resonance Imaging (MRI) technology. However, without being limited thereto, the brain images may include images captured based on various technologies, such as Computed Tomography (CT), Positron Emission Tomography (PET), Single Photon Emission Computed Tomography (SPECT), or Electroencephalography (EEG).

In step 303, the processor 110 may partition (or segment) the brain images into a plurality of predefined analysis target regions according to anatomical criteria.

According to one embodiment, the processor 110 may partition the brain images into regions including a tight high-convexity region of the cerebrum, a region encompassing enlarged Sylvian fissures, a region exhibiting ventriculomegaly, and an intracranial region.

The processor 110 may label the partitioned analysis target regions in the region-partitioned brain images, and based on this, the brain images may provide information on the partitioned regions.

According to one embodiment, the tight high-convexity region of the cerebrum is located near the surface of the brain, where neural tissue is relatively dense. This region has unique structural characteristics, and thus may exhibit a relatively high signal intensity in brain images (e.g., MRI images). High signal intensity generally occurs when tissue density is high or when physical properties differ. In brain imaging, neural tissue is composed of various substances such as water, fat, and protein, and the signal intensity in brain images may vary depending on the characteristics of each substance.

In particular, the tight high-convexity region of the cerebrum is located near the surface of the brain, and due to the high-density neural tissue, which is distinct from the cerebrospinal fluid (CSF) that exhibits a relatively low signal, a high signal may be reflected in the brain images.

The density (or tissue density) that affects signal intensity in brain images may refer to the density or distribution of specific substances contained in the tissue. For example, in the case of MRI, density may be determined based on the density or distribution of hydrogen atoms contained in the tissue, and in the case of CT, CT image density is determined by electron density, elemental composition and spatial distribution, and physical density.

Herein, the signal intensity appearing through the brain images may be set as a pixel value in the image. More specifically, pixels in brain images have a value of a specific hue (e.g., grayscale), and it can be explained that the closer to a bright color (e.g., white), the higher the signal intensity, and the closer to a dark color (e.g., black), the lower the signal intensity. In addition, if the signal intensity of a pixel in a brain image is high, it may indicate that the signal emitted from the tissue of the corresponding pixel is strong and that the tissue has specific physical characteristics. Conversely, if the signal intensity of a pixel in a brain image is low, it may indicate that the signal emitted from the tissue of the corresponding pixel is weak and that the tissue has different physical or chemical characteristics.

Also, the tight high-convexity region of the cerebrum has a convex shape on the surface of the brain, so the curvature of this region has a characteristic that distinguishes it from other regions. The tight high-convexity region of the cerebrum is located in the upper part of the brain, and its surface may appear relatively smooth and curved. This morphological characteristic appears as a part with high curvature in MRI images, and it may show a clear difference when compared to other flat regions of the brain.

Based on this, the processor 110 may partition the tight high-convexity region of the cerebrum or partition the boundary of the tight high-convexity region of the cerebrum based on a predetermined reference signal value (or signal range) and/or a predetermined reference curvature value (or curvature range) for the tight high-convexity region of the cerebrum in the brain images. Herein, according to one embodiment of the curvature, it can be described as having a unit of 1/mm as the reciprocal of the radius of curvature (R).

The processor 110 may set a normalized intensity based on the brain imaging system and the anatomical characteristics of the tissue, and determine the signal intensity of the pixels in the brain images as a relative value according to the normalized intensity. However, without being limited thereto, the processor 110 may determine the signal intensity of the pixels based on a signal intensity unit (SIU).

In addition, the predetermined reference values (or ranges) related to the partitioning of the tight high-convexity region of the cerebrum may be set by an expert (e.g., a medical professional such as a neuroimager).

According to one embodiment, identifying the region of enlarged Sylvian fissures involves identifying the expanded portion of the Sylvian fissure in the brain image. The Sylvian fissure is the main fissure of the brain that separates the temporal lobe and the parietal lobe, and when this region is enlarged, it may be related to structural abnormalities of the brain or specific diseases of the brain.

In the case of MRI images, the region of enlarged Sylvian fissures can be distinguished by two main characteristics. First, the Sylvian fissure is generally located on the side of the brain, and when this region is enlarged, it may appear abnormally wide in brain images. Second, the Sylvian fissure may have characteristic boundaries and signal patterns that distinguish it from other major structures of the brain. For example, this region may exhibit a relatively low signal intensity unlike the surrounding brain tissue, and in particular, it may exhibit characteristics similar to the signal (e.g., inherent signal) set for cerebrospinal fluid (CSF).

Accordingly, the processor 110 may accurately detect the boundary of the Sylvian fissure and the location of the Sylvian fissures in the brain image based on a predetermined reference signal value (or signal range).

According to one embodiment, the ventriculomegaly region is a space inside the brain where cerebrospinal fluid (CSF) flows, and it has unique structural characteristics that distinguish it from neural tissue. The ventriculomegaly region exhibits a unique signal pattern in MRI images, and the size and shape of the ventricles can provide important information for evaluating the functional and structural state of the brain. The ventricles are physically located in the space across the center of the brain, and the cerebrospinal fluid may have a characteristic of exhibiting a relatively low signal intensity.

The ventricles are largely divided into the first and second lateral ventricles, the third ventricle, and the fourth ventricle, each of which can be clearly distinguished in MRI images. The first and second lateral ventricles are located on the sides of the cerebral hemispheres and fill the space of the cerebral hemispheres. This region contains cerebrospinal fluid, which exhibits a relatively low signal intensity, making it easily distinguishable in MRI images. The third ventricle runs across the center of the brain and plays an important role in connecting the left and right sides of the brain. The fourth ventricle is located in the continuous part of the brainstem and spinal cord and may regulate the circulation of cerebrospinal fluid between the brain and spinal cord.

In the case of MRI images, the ventriculomegaly region can be distinguished by two main characteristics. First, the ventricles are located in the center of the brain, and this region appears as an empty space occupied by cerebrospinal fluid. Since cerebrospinal fluid exhibits a relatively low signal intensity, this part forms a characteristic region that is distinguished from the surrounding brain tissue. Second, the boundaries of the ventricles may exhibit a relatively distinct signal pattern unlike other major structures of the brain. For example, the first and second lateral ventricles may have boundaries that are distinguishable from other brain structures because they are located on the sides of the brain.

The size and shape of the ventricles may show abnormal changes in patients with diseases compared to normal anatomical structures, and this can be used as an indicator of brain disease. For example, enlargement or abnormal shape of the ventricles may indicate conditions such as hydrocephalus or brain atrophy. Based on this, the processor 110 may accurately identify the location and boundary of the ventricles by comparing and judging the signal intensity of the pixels of the tissue based on a predetermined reference signal value (or signal range).

According to one embodiment, the intracranial region is a bone structure that protects the brain, forming an anatomical structure surrounding the outside of the brain, and the brain and skull are closely related. The skull is made up of several bones, which protect the brain from external impact. The skull is composed of high-density bone tissue, so the density is very high. Therefore, in the case of MRI images, the skull exhibits characteristic strong signal intensity and structural characteristics that distinguish it from other tissues of the brain.

In partitioning the intracranial region, a predetermined reference signal value (or signal range) may be set to reflect the anatomical characteristics related to the characteristics of the skull. For example, the reference signal value (or signal range) set to partition the intracranial region may be set to a higher value (or range) than the reference signal value (or signal range) set to partition the tight high-convexity region of the cerebrum. Through this, the processor 110 may accurately identify the intracranial region in the MRI image and clearly distinguish the boundary between the brain and the skull.

According to another embodiment, the processor 110 may input the patient's brain images to a region partitioning module 201 that is trained to partition the analysis target regions (tight high-convexity region of the cerebrum, region of enlarged Sylvian fissures, ventriculomegaly region, and intracranial region) of the brain images, and from the region partitioning module 201, and the processor 110 may receive, from the region partitioning module 201, brain images in which the tight high-convexity region of the cerebrum, the region encompassing enlarged Sylvian fissures, the ventriculomegaly region, and the intracranial region are partitioned (or labeled).

According to the above description, the processor 110 was described as labeling the analysis target regions partitioned in the brain images from the region partitioning module 201 or acquiring brain images in which the partitioned analysis target regions are labeled.

However, the processor 110 may generate separate images for each of the analysis target regions partitioned from the brain images and input them to the region partitioning module 201, and the region partitioning module 201 may partition the analysis target regions from the input brain images, partition each of the partitioned regions, and generate and output a separate image.

In step 305, the processor 110 may extract feature information for detecting the state of the disease from the plurality of analysis target regions partitioned in the brain images.

According to one embodiment, the processor 110 may analyze at least some feature information of volume, shape, signal intensity, and texture for each analysis target region in the brain images partitioned (or segmented) into the tight high-convexity region of the cerebrum, the region encompassing enlarged Sylvian fissures, the ventriculomegaly region, and the intracranial region, and may extract information that is characteristic of each analysis target region.

According to one embodiment, based on the pixel data of the tight high-convexity region of the cerebrum, the region of enlarged Sylvian fissures, the ventriculomegaly region, and the intracranial region partitioned in the brain images, the processor 110 may extract various feature information reflecting the physical and anatomical characteristics of each analysis target region.

More specifically, first, the processor 110 may analyze the pixel data of each partitioned analysis target region to extract the volume feature of the corresponding analysis target region.

The processor 110 may calculate the volume of the corresponding region by three-dimensionally analyzing the number of pixels within each analysis target region of the brain images. For example, the processor 110 may check the number of pixels in the ventriculomegaly region from the brain images in which the ventriculomegaly region is partitioned, and based on this, calculate the volume of the ventriculomegaly region.

The processor 110 may apply 3D volume rendering technology and/or Voxel-based morphometry (VBM) to three-dimensionally analyze the volume for each analysis target region from the brain images. In addition, the processor 110 may perform a comparative analysis with a predetermined normal brain volume value, detect a location where the volume difference exceeds a specific criterion, label information about the difference identified at the location, and use it in a later process.

In addition, the processor 110 may analyze the boundary and shape of each partitioned analysis target region to extract shape features such as asymmetry, sphericity, and curvature.

For example, the processor 110 may calculate the curvature of each analysis target region based on a characteristic radius of curvature (R) (e.g., predetermined radius of curvature (R) for each analysis target region) for the boundary of each analysis target region. In addition, the processor 110 may detect locations with a radius of curvature value below a predetermined reference value (e.g., a reference value set for the corresponding region of a normal brain), and label information about the difference identified at those locations (e.g., the possibility of cortical atrophy in the case of the tight high-convexity region of the cerebrum).

The processor 110 may apply Fractal Analysis, Edge Detection, or 3D mesh modeling techniques to perform shape analysis of each analysis target region based on the brain images.

In addition, the processor 110 may analyze the signal intensity of pixels in each partitioned analysis target region to extract the density and physical characteristics of specific tissues. Here, the processor 110 may detect the signal intensity for each analysis target region in a similar manner to the operation of detecting the signal intensity from the brain images in order to partition the analysis target regions from the brain images.

At this time, the processor 110 may calculate the average, median, or standard deviation of the signal intensities of the pixels for each of the analysis target regions and determine it as the signal intensity of the corresponding analysis target region. In addition, the processor 110 may label information about the difference if the calculated signal intensity value exceeds the difference between a predetermined reference value (e.g., a reference value set for the corresponding region of a normal brain) and a predetermined comparison reference value.

In addition, the processor 110 may apply various texture analysis methods such as GLCM, LBP, and wavelet transform techniques to each partitioned analysis target region to extract tissue patterns and spatial changes in signals.

For example, the processor 110 may measure the homogeneity and contrast of the signal through Gray-Level Co-occurrence Matrix (GLCM)-based texture analysis in the region of enlarged Sylvian fissures.

The processor 110 may also apply Local Binary Pattern (LBP), Wavelet Transformation, etc. to detect minute structural differences from a predetermined normal reference value (e.g., a reference texture value of the normal cortex predetermined for the region of enlarged Sylvian fissures of a normal brain), and label information about the differences.

After that, the processor 110 may store the extracted feature information of volume, shape, signal intensity, and texture in a database, and perform preprocessing and statistical evaluation for subsequent analysis.

For example, before generating a biomarker for determining (or used for determining) a brain disease such as normal pressure hydrocephalus or Alzheimer's-type dementia, the processor 110 may perform various preprocessing and statistical evaluations using the feature information of volume, shape, signal intensity, and texture extracted from the brain images.

The processor 110 may normalize and standardize the extracted feature information. Since the signal intensity and tissue density of each brain image may differ depending on the settings of the imaging device and environmental factors, the processor 110 may normalize the feature information and convert it into a comparable range.

According to one embodiment, the processor 110 may apply Min-Max scaling to adjust all feature values to values between 0 and 1, or apply Z-score standardization to adjust the mean of the data to 0 and the standard deviation to 1. These normalization and standardization processes may contribute to maintaining the consistency of the data and increasing the accuracy in the subsequent analysis process.

Also, the processor 110 may detect and handle outliers. For example, in the case where some feature values deviate from the normal range due to noise or imaging errors in the MRI image, the processor 110 may perform a box plot-based interquartile range (IQR) analysis to identify and remove or replace outlier values.

In addition, the processor 110 may apply Mahalanobis Distance-based analysis to detect outliers in high-dimensional data, and if necessary, may adjust extreme values within a threshold range through the Winsorization technique.

Also, the processor 110 may handle missing values to ensure the integrity of the data. Since the signal in a specific region in the brain image may not be clear or data may be lost during the analysis process, the processor 110 may apply a replacement method using the mean or median value, or predict missing feature information through the K-Nearest Neighbors (KNN) algorithm and complement it.

Also, the processor 110 may apply Multiple Imputation by Chained Equations (MICE) to estimate missing data in complex patterns.

In addition, the processor 110 may reduce the data dimension of the feature information. For the extracted feature information of volume, shape, signal intensity, and texture, the processor 110 may apply Principal Component Analysis (PCA) to select features that have a high contribution to disease determination according to predetermined criteria and remove variables with low contribution.

In addition, the processor 110 may utilize Linear Discriminant Analysis (LDA) to transform the data to maximize the variance between predetermined classes and minimize the variance within the classes for the discrimination of normal and specific diseases (e.g., normal pressure hydrocephalus and Alzheimer's-type dementia). Through this, it is possible to more clearly distinguish the feature differences between normal and abnormal states, thereby improving the accuracy of disease discrimination.

In addition, the processor 110 may apply Gaussian filtering or Wavelet Transformation to remove noise from the image, thereby reducing noise that may occur during the signal intensity and texture analysis process. This noise removal technique plays an important role, especially in low-resolution MRI images, and may contribute to improving accuracy in the analysis process.

In addition, the processor 110 may apply a clustering technique to analyze the pattern of the data. For example, the processor 110 may group normal and abnormal patterns using the K-Means Clustering or Density-Based Spatial Clustering of Applications with Noise (DBSCAN) algorithm, and analyze the differences between each group. Through this clustering technique, the processor 110 may effectively distinguish normal and abnormal patterns and systematically organize the data so that it can be used in the subsequent disease discrimination process.

In addition, the processor 110 may perform statistical evaluation using the preprocessed feature information. For this purpose, correlation analysis may be applied to evaluate the association between specific features and to statistically verify the difference between normal and abnormal states. In addition, ANOVA (analysis of variance) or t-test may be performed to confirm significant differences between groups and to quantify the effect of feature information on disease discrimination.

According to another embodiment, the processor 110 may input the brain images with the analysis target regions partitioned to the feature extraction module 203, which is trained to extract feature information for each of the analysis target regions from the brain images with the analysis target regions partitioned, and may obtain, from the feature extraction module 203, feature information extracted for each of the analysis target regions or brain images with the feature information labeled.

In step 307, the processor 110 may generate a biomarker for determining the state of the disease from the feature information extracted from the brain images in at least one of the analysis target regions of the brain images, based on the pre-trained brain image processing model 123.

More specifically, the processor 110 may input the feature information extracted for each of the analysis target regions of the brain images to the biomarker generation module 205 constituting the brain image processing model 123.

However, without being limited thereto, the processor 110 may input the feature information and the brain images in which the analysis target regions are partitioned to the biomarker generation module 205.

Based on the input data, the biomarker generation module 205 may generate a biomarker for at least some of the analysis target regions, and output at least one brain image in which the biomarker is displayed.

For example, the biomarker generation module 205 may be configured to generate a biomarker for each category of feature information (e.g., volume, shape, signal intensity, or texture) (e.g., a volume-based biomarker, a shape-based biomarker, a signal intensity-based biomarker, or a texture-based biomarker).

More specifically, for example, the biomarker generation module 205 may generate biomarkers such as Ventricular Volume Ratio (VVR) or Cortical Volume Reduction Index (CVRI) for determining brain disease based on the volume feature information extracted for the analysis target regions.

In addition, the biomarker generation module 205 may generate biomarkers such as an asymmetry index or surface complexity for determining brain disease based on the shape feature information such as curvature and asymmetry extracted for the analysis target regions.

In addition, the biomarker generation module 205 may generate a biomarker such as a normalized signal intensity ratio (NSIR) for determining a brain disease based on the signal sensitivity feature information extracted for the analysis target regions.

In addition, the biomarker generation module 205 may generate biomarkers such as feature pattern irregularity or texture complexity for determining brain disease based on the texture feature information extracted for the analysis target regions.

In addition, the brain image output by the biomarker generation module 205 may be configured to include an image for each of the analysis target regions, an image for two or more of the analysis target regions, or an image in which the analysis target regions are partitioned (labeled).

In step 309, the processor 110 may determine the patient's state based on the biomarker generated through the brain image processing model 123.

More specifically, the processor 110 may analyze the biomarker and/or feature information generated in step 307 using the brain image processing model 123, and based on this analysis, determine if the patient's state corresponds to normal pressure hydrocephalus, Alzheimer's disease, or a normal condition.

The processor 110 may determine the patient's brain disease state by analyzing the quantitative value of the biomarker and comparing it with a predetermined normal reference. The processor 110 may individually evaluate or comprehensively review the volume, shape, signal intensity, and texture feature information extracted from each analysis target region in order to analyze the quantitative value of the generated biomarker.

For example, the processor 110 may determine that the patient has normal pressure hydrocephalus (or has an increased possibility of normal pressure hydrocephalus) when the ventricular volume ratio (VVR) is higher than the normal reference and the cortical volume reduction index (CVRI) is lower than the normal reference. On the other hand, the processor 110 may determine that it is Alzheimer's-type dementia (or has an increased possibility of Alzheimer's-type dementia) when the Asymmetry Index (AI) and Surface Complexity (SC) are higher than the normal reference.

In addition, the processor 110 can determine disease presence by comparing generated biomarker values against pre-set thresholds for normal and diseased states.

For example, with a threshold value of 0.4 for the Ventricular Volume Ratio (VVR), the processor 110 may determine normal pressure hydrocephalus (or a high likelihood of NPH) if the VVR exceeds this value, and a normal state (or a low likelihood of NPH) if it is lower. Such threshold values may be established based on large-scale clinical data acquired by the processor 110 and may be refined based on the diagnostic expertise of medical professionals.

Additionally, the processor 110 may predict the disease state and progression based on biomarkers and feature information. For example, the processor 110 may perform a quantitative analysis by applying predetermined reference values and clinical guidelines, and evaluate the interrelationship between biomarkers to determine the progression state of the disease.

More specifically, the processor 110 may individually evaluate the biomarker values extracted from each analysis target region and compare them to predetermined reference values for normal and/or diseased states (e.g., normal pressure hydrocephalus, Alzheimer's disease) to assess the patient's condition. For example, the processor 110 may determine the patient's state as normal if the Ventricular Volume Ratio (VVR) is 0.3 or less, borderline if between 0.3 and 0.4, and normal pressure hydrocephalus (or a high likelihood thereof) if 0.4 or greater. These reference values may be established based on large-scale clinical data acquired by the processor 110 and adjusted according to medical guidelines.

Furthermore, the processor 110 may analyze the correlation between multiple biomarkers to predict changes in the disease state. For instance, by analyzing the combination of the Cortical Volume Reduction Index (CVRI) and the Ventricular Volume Ratio (VVR), the processor 110 can assess a high likelihood of Alzheimer's disease progression if both values increase simultaneously. By evaluating whether the combination of two or more biomarkers matches the characteristic pattern of a specific disease, the processor 110 can predict not only the patient's current state but also the likelihood of future disease progression.

Additionally, the processor 110 may predict the rate of disease progression by analyzing the rate of change of biomarkers. For example, if the rate of increase in the Ventricular Volume Ratio (VVR) over a recent predetermined period is faster than that of normal aging, the processor 110 may determine a rapid progression of normal pressure hydrocephalus.

To perform this type of trend analysis, the processor 110 may compare and analyze brain image data acquired from the same patient at predetermined time intervals (e.g., 3 weeks, 1 month, etc.), thereby assessing the stage of disease progression by comparing the patient's previous state with their current state.

Moreover, the processor 110 may perform a stage-by-stage classification of the disease based on specific clinical criteria. For example, in the case of normal pressure hydrocephalus, the early stage may show mild ventricular enlargement but minimal cortical atrophy, whereas the advanced stage may be accompanied by changes in signal intensity along with ventricular enlargement. The processor 110 may analyze these patterns to determine whether the patient is in an early, intermediate, or advanced stage.

According to various embodiments, the processor 110 may input the biomarkers and feature information into a machine learning-based brain disease determination module 207 to predict the patient's brain disease state and the degree of progression.

For instance, the trained brain disease determination module 207 can compare the extracted feature information and/or the biomarkers generated based on the extracted feature information with learned patterns and predict the risk of progression of normal pressure hydrocephalus and Alzheimer's disease. This model is configured to utilize past assessment data to enable early detection of disease and numerical assessment of its progression.

The processor 110 may end the embodiment of FIG. 3 upon determining the patient's brain disease state and/or the stage of progression of the brain disease through step 307.

Furthermore, according to various embodiments, the processor 110 may visually represent the determined patient status. For example, the processor 110 may overlay and label the brain images with a color code indicating the level of disease risk (e.g., normal = green, mild abnormality = yellow, severe = red).

Furthermore, the processor 110 may output quantitative risk likelihood based on the numerical data; for example: an 85% likelihood of normal pressure hydrocephalus, a 10% likelihood of Alzheimer's disease, and a 5% likelihood of being in a normal state.

Moreover, the processor 110 may visually represent the analysis results to support healthcare professionals in intuitively understanding the disease state. For example, the processor 110 may output biomarker values for each analysis target region in the form of a graph or a heatmap, allowing visual confirmation of the degree of abnormalities occurring in specific regions. This enables healthcare professionals to quickly grasp the patterns of biomarker changes and make prompt decisions about the patient's condition.

In this manner, the processor 110 may utilize the generated various biomarkers and feature information to comprehensively assess the patient's condition, improve the accuracy of differentiating between normal pressure hydrocephalus and Alzheimer's disease, and support early assessment and treatment planning for the disease.

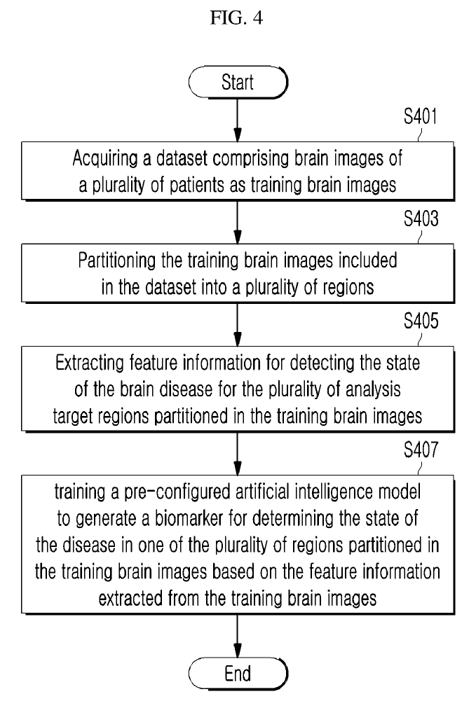

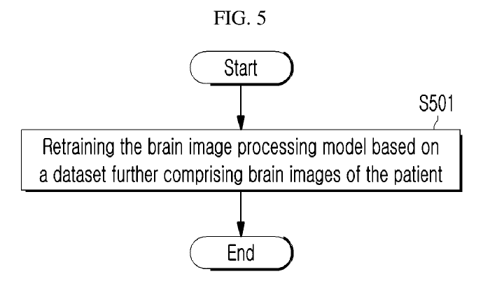

Hereinafter, the training of the brain image processing model 123, including the brain disease determination module 207, will be described with reference to FIGS. 4 and 5. In this regard, FIG. 4 is a flowchart illustrating a flow of operations for training a brain image processing model for assessing a brain disease state in an apparatus according to one embodiment. FIG. 5 is a flowchart illustrating a flow of operations for re-training a brain image processing model for assessing a brain disease state in an apparatus according to one embodiment.

First, referring to FIG. 4, in step 401, the processor 110 may generate a dataset including brain images of a plurality of patients as training brain images. Herein, the processor 110 may acquire a plurality of brain images from the training image data 121 of the memory 120. The processor 110 may store the generated dataset in the memory 120.

Herein, at least some of the brain images stored in the training image data 121 may have the analysis target regions partitioned, or may include images for at least one analysis target region. In addition, at least some of the brain images stored in the training image data 121 may have a biomarker generated for at least one analysis target region. Furthermore, at least some of the brain images stored in the training image data 121 may have the brain disease state of the corresponding patient (e.g., normal pressure hydrocephalus, Alzheimer's disease, or normal state) labeled.

In step 403, the processor 110 may partition the training brain images into a plurality of analysis target regions. For example, the processor 110 may partition the analysis target regions in the training brain images based on at least some of the operations of partitioning the brain images into a plurality of analysis target regions in step 303.

However, if the brain image processing model 123 includes a trained region partitioning module 201, the processor 110 may input the training brain images to the region partitioning module 201 and obtain, from the region partitioning module 201, brain images with the analysis target regions partitioned.

Herein, the brain images with the analysis target regions partitioned may be brain images for at least one analysis target region and/or brain images with the analysis target regions labeled.

To this end, the region partitioning module 201 may be in a trained state to partition the analysis target regions from the input brain images and/or to generate brain images with the analysis target regions partitioned.

Hereinafter, the training of the region partitioning module 201 may be described.

According to one embodiment, the processor 110 may normalize the signal intensity of the training brain images to reduce contrast differences in the images and unify the input size of the model through resizing. Additionally, the processor 110 may establish a region of interest (ROI) focused on the analysis target region within the training brain images where analysis target regions have been partitioned.

This preprocessing supports effective training of the region partitioning module 201 on key features, maintains uniform quality of the training images, and ensures training consistency.

After that, the processor 110 may train the region partitioning module 201 based on supervised learning techniques and/or unsupervised learning techniques.

According to one embodiment, in the case of supervised learning, the processor 110 may train the region partitioning module 201 by processing the training brain images and ground truth labels in which the analysis target regions are identified for the training brain images as input.

Herein, the processor 110 may train the region partitioning module 201 by applying deep learning networks such as U-Net, Fully Convolutional Network (FCN), and DeepLab. In addition, the processor 110 may proceed with the training of the region partitioning module 201 by using a loss function (e.g., cross-entropy, Dice loss) to minimize the error between the input image and the ground truth label.

In addition, the processor 110 may create an optimal learning environment through learning rate adjustment and setting the number of iterations (epochs), and may perform model evaluation of the region partitioning module 201 using validation data to achieve a performance level above a certain level.

According to another embodiment, in the case of unsupervised learning, the processor 110 may train the region partitioning module 201 by including brain images collected without ground truth labels in the training brain images.

The processor 110 may apply clustering-based algorithms (e.g., K-means clustering, hierarchical clustering) among unsupervised learning methods, and through these techniques, the processor 110 may train the region partitioning module 201 to partition the analysis target regions based on the pixel distribution and similar patterns in the brain images.

In addition, the processor 110 may apply deep learning techniques such as Autoencoder to compress the high-dimensional features of the brain images into low dimensions, and then train the region partitioning module 201 to automatically classify regions with similar features.

In addition, the processor 110 may perform training of the region partitioning module 201 by applying statistical characteristics of the image (e.g., mean and variance of signal intensity), spatial patterns of tissue (e.g., texture analysis), edge detection, etc. to increase the accuracy of region partitioning through unsupervised learning.

In addition, the processor 110 may perform training of the region partitioning module 201 by applying a semi-supervised learning technique that automatically generates ground truth labels in the unsupervised learning process. In this method, the remaining data is predicted based on some given correct answer data, and learning is performed by continuously improving the generated prediction results.

In this way, the processor 110 may perform training of the region partitioning module 201 by concurrently or independently applying supervised learning and unsupervised learning techniques.

In step 405, the processor 110 may extract feature information for detecting the state of the brain disease for the plurality of analysis target regions partitioned in the training brain images.

For example, the processor 110 may extract feature information for each analysis target region in the training brain images based on at least some of the operations of extracting feature information for detecting the state of the disease from the partitioned analysis target regions of the brain images described in step 305.

However, if the brain image processing model 123 includes a trained feature extraction module 203, the processor 110 may input the training brain images with the analysis target regions partitioned to the feature extraction module 203 and the feature extraction module 203 may obtain data with the feature information extracted for each analysis target region. This data may include feature information on volume, shape, signal intensity, and texture for at least one analysis target region, and/or brain images with the feature information labeled.

To this end, the feature extraction module 203 may be in a trained state to extract feature information for each analysis target region from the input brain images and/or to generate brain images from which feature information has been extracted.

Hereinafter, the training of the feature extraction module 203 may be described.

According to one embodiment, the processor 110 may normalize the signal intensity of the training brain image to reduce contrast differences in the image and unify the input size of the model by resizing. In addition, the processor 110 may set a Region of Interest (ROI) in the training brain image in which the analysis target region is partitioned to increase the accuracy of feature information extraction.

Through this preprocessing, it is possible to support the feature extraction module 203 to effectively learn important features, maintain the quality of the training images uniformly, and ensure consistency of learning.

After that, the processor 110 may train the feature extraction module 203 based on supervised learning techniques and/or unsupervised learning techniques.

According to one embodiment, in the case of supervised learning, the processor 110 may train the feature extraction module 203 by processing the training brain images and ground truth labels including the feature information of the analysis target regions for the training brain images as input.

Herein, the processor 110 may train the feature extraction module 203 by applying deep learning networks such as CNN, ResNet, and DenseNet. In addition, the processor 110 may proceed with the training of the feature extraction module 203 by using a loss function (e.g., mean squared error, cross-entropy loss) to minimize the error between the input image and the ground truth label.

In addition, the processor 110 may create an optimal learning environment through learning rate adjustment and setting the number of iterations (epochs), and may perform model evaluation of the feature extraction module 203 using validation data to achieve a performance level above a certain level.

According to another embodiment, in the case of unsupervised learning, the processor 110 may train the feature extraction module 203 by including brain images collected without ground truth labels in the training data. The processor 110 may apply clustering-based algorithms (e.g., K-means clustering, hierarchical clustering) among unsupervised learning methods, and through these techniques, the processor 110 may train the feature extraction module 203 to extract the feature information of the analysis target region based on the pixel distribution and similar patterns in the brain images.

In addition, the processor 110 may apply Autoencoder and Principal Component Analysis (PCA) to compress the high-dimensional features of the training brain images into low dimensions, and then train the feature extraction module 203 to automatically classify regions with similar features.

In addition, the processor 110 may perform training of the feature extraction module 203 by applying statistical characteristics of the image (e.g., mean and variance of signal intensity), spatial patterns of tissue (e.g., texture analysis), edge detection, etc. to increase the accuracy of feature extraction through unsupervised learning.

In addition, the processor 110 may perform training of the feature extraction module 203 by applying a semi-supervised learning technique that automatically generates ground truth labels in the unsupervised learning process. In this method, the remaining data is predicted based on some given correct answer data, and learning is performed by continuously improving the generated prediction results.

In this way, the processor 110 may perform training of the feature extraction module 203 by concurrently or independently applying supervised learning and unsupervised learning techniques.

In step 407, the processor 110 may train the biomarker generation module 205 of the brain image processing model 123 to generate a biomarker for determining the state of the disease in one of the plurality of regions partitioned in the training brain images based on the feature information extracted from the training brain images. More specifically, the biomarker generation module 205 may be trained to generate a biomarker for determining the state of normal pressure hydrocephalus (NPH) and Alzheimer's-type dementia (AD) based on the volume feature information of the analysis target region of the training brain image. Changes in volume in specific regions of the brain can be a major indicator of disease, and by quantitatively analyzing this, a criterion for disease discrimination can be prepared.

For example, the biomarker generation module 205 may be trained to generate the ventricular volume ratio (VVR) as a biomarker. The biomarker module 205 may be trained to evaluate whether the ventricles are enlarged by calculating the size of the ventricles as a ratio to the intracranial volume based on the ventricular volume ratio. The biomarker generation module 205 may be trained to calculate the volume based on the number of pixels in the ventricular region and the intracranial region in the brain image, and quantitatively compare them to generate the ventricular volume ratio (VVR) value.

In addition, the biomarker generation module 205 may be trained to generate the cortical volume reduction index (CVRI) as a biomarker. CVRI can be utilized to evaluate the progression of Alzheimer's-type dementia by comparing the volume of the tight high-convexity region of the cerebrum with the normal cerebral volume. In the case of Alzheimer's-type dementia, the cerebral cortex tends to atrophy, and by numerically measuring this change, it is possible to determine whether or not there is a disease early.

In addition, the biomarker generation module 205 may be trained to generate a biomarker for evaluating the state of normal pressure hydrocephalus and Alzheimer's-type dementia based on the shape feature information extracted from the analysis target region. Structural changes in the brain are closely related to the progression of disease, and by quantitatively analyzing them, reliable criteria can be provided.

In this regard, the biomarker generation module 205 may be trained to generate an asymmetry index (AI) as a biomarker. The cerebrum normally has a symmetrical structure on the left and right, but asymmetry may increase as the disease progresses. The biomarker generation module 205 may be trained to calculate the volume of each analysis target region and then quantitatively analyze the difference between the left and right regions to set it as a biomarker.

In addition, the biomarker generation module 205 may be trained to generate surface complexity (SC) as a biomarker. The surface shape of the superior cerebrum and ventricles may be simplified or show abnormal protrusion patterns as the disease progresses. To evaluate this, the biomarker generation module 205 may be trained to measure the curvature radius and quantify the surface complexity by applying Fractal Analysis and 3D mesh modeling.

In addition, the biomarker generation module 205 may be trained to generate the Sylvian Fissure Enlargement Index (SFEI) as a biomarker. The degree of enlargement of the Sylvian fissure is one of the main characteristics of normal pressure hydrocephalus, and it can be an important indicator for evaluating whether or not cerebrospinal fluid (CSF) has increased. The biomarker generation module 205 may be trained to extract the boundary of the Sylvian fissure, measure the area of the corresponding region, and compare it with a normal reference value to determine whether or not it is enlarged.

In addition, the biomarker generation module 205 may be trained to generate a biomarker for determining the state of normal pressure hydrocephalus (NPH) and Alzheimer's-type dementia (AD) based on the signal intensity feature information extracted from the analysis target regions. For this purpose, the biomarker generation module 205 may be trained to measure signal intensities at the pixel level within the analysis target regions and to generate, based on these measurements, quantitative metrics that can distinguish between normal and abnormal states.

For example, the biomarker generation module 205 may be trained to generate a biomarker of normalized signal intensity ratio (NSIR). NSIR may be used to evaluate the relative signal intensity of a specific analysis target region by comparing the average signal intensity of the specific analysis target region with normal reference data. The biomarker generation module 205 may be trained to calculate the average signal intensity of the Sylvian fissure region and compare it with the reference value of a normal person to evaluate whether the signal intensity has increased or decreased.

In addition, the biomarker generation module 205 may be trained to generate a biomarker of the tissue signal intensity variation index (TSIVI). TSIVI is a biomarker that evaluates the variability of signal intensity between pixels within a specific analysis target region, and may be utilized to evaluate the uniformity and physical characteristics of brain tissue. The biomarker generation module 205 may be trained to analyze the signal intensity of the tight high-convexity region of the cerebrum, and calculate the standard deviation and coefficient of variation to detect abnormal signal intensity changes compared to normal.

The biomarker generation module 205 may generate biomarkers for determining the state of disease based on texture feature information as well as signal intensity. For example, the biomarker generation module 205 may be trained to analyze fine patterns and spatial signal changes in tissue within the analysis target region.

The biomarker generation module 205 may generate a texture uniformity index (THI) as a biomarker. THI may be used to evaluate the texture pattern between pixels in the analysis target region using Gray-Level Co-occurrence Matrix (GLCM) and measure the spatial uniformity of the signal.

In addition, the biomarker generation module 205 may be trained to generate a texture complexity index (TCI) as a biomarker. TCI is a biomarker that evaluates the complexity of the fine pattern of tissue by applying Local Binary Pattern (LBP) and Wavelet Transformation techniques.

The biomarker generation module 205 may also be trained to generate biomarkers for determining brain disease by comprehensively analyzing various feature information such as volume, shape, signal intensity, and texture extracted from the analysis target region.

To this end, the processor 110 may normalize the feature information, adjust the unit differences, and transform it so that multidimensional analysis is possible. The processor 110 may perform normalization and standardization to adjust features such as volume, shape, signal intensity, and texture, which have different ranges, to the same standard.

For example, since the ventricular volume ratio (VVR) and the normalized signal intensity ratio (NSIR) have different units, the processor 110 may convert them to a range of 0 to 1 so that they can be compared. In addition, the processor 110 may apply principal component analysis (PCA) or linear discriminant analysis (LDA) to construct a multidimensional feature vector and analyze the pattern of the disease.

Based on this, the biomarker generation module 205 may be trained to generate various biomarkers for the discrimination of brain diseases. For example, the biomarker generation module 205 may be trained to generate a Global Brain Structure Anomaly Index (GSAI) by integrating volume and shape information. This can be utilized to evaluate the overall structural abnormality of the brain by integrating factors such as ventricle size, cortical volume reduction, and shape asymmetry.

In addition, the biomarker generation module 205 may generate a Composite Disease Differentiation Index (CDDI) by combining signal intensity and texture information extracted from the analysis target region. This index is used as a key indicator for the differentiation of normal pressure hydrocephalus and Alzheimer's-type dementia, and may enable more accurate assessment by reflecting the signal intensity pattern and tissue texture differences between the two diseases.

The training of the biomarker generation module 205 may be performed based on the configuration of the biomarker generation module 205 and the control of the processor 110.

After completing the operations of step 407, the processor 110 may terminate the process described in the embodiment of FIG. 4.

Returning to FIG. 3, after performing the operation of step 309, the processor 110 may perform retraining of the biomarker generation module 205 as shown in FIG. 5.

In step 501, the processor 110 may retrain the brain image processing model based on a dataset further including brain images of the patient.

More specifically, the processor 110 may perform retraining of the brain image processing model 123 by including the brain images used to determine the patient's brain disease state in the dataset on which the training of the brain image processing model 123 was performed.

According to one embodiment, the processor 110 may perform retraining of the brain image processing model 123 based on at least some of the operations of training the brain image processing model 123 of FIG. 4.

However, without being limited thereto, the processor 110 may apply a transfer learning technique to effectively reflect the influence of new data while maintaining the performance of the existing model. Through this, the processor 110 may achieve performance improvement more efficiently than retraining the modules constituting the brain image processing model 123 from scratch.

For example, the processor 110 may prevent overfitting of the brain image processing model 123 by freezing the biomarker generation module 205 and fine-tuning only specific weights based on the additional data. This can be combined with regularization techniques to enhance generalization performance.

In addition, the processor 110 may apply a data augmentation technique in the retraining operation. For example, the processor 110 may apply various transformations of the brain images (e.g., rotation, translation, contrast adjustment).

Through this, the processor 110 may support the brain image processing model 123 to learn various changes in the data and maintain high accuracy even in a new environment.

According to various embodiments, by providing a method and apparatus for determining a patient's brain disease state from brain images, normal pressure hydrocephalus and Alzheimer's-type dementia can be rapidly and accurately distinguished, thereby reducing assessment time and providing patients with faster treatment planning.

For example, a treatment for normal pressure hydrocephalus includes, but not limited to, a surgery of placing a tube, called a shunt, into the brain to drain the excess fluid, and an endoscopic third ventriculostomy (ETV) which is a minimally invasive surgical procedure that creates an opening in the floor of the third ventricle to allow cerebrospinal fluid (CSF) to escape and bypass a blockage, effectively treating hydrocephalus.

For example, a treatment for Alzheimer's-type dementia includes, but not limited to, a medicine including cholinesterase Inhibitors such as donepezil, galantamine, and rivastigmine, memantine, aducanumab, lecanemab, and/or donanemab. The treatment may be a non-drug therapy such as cognitive stimulation therapy (CST) and cognitive rehabilitation.

According to various embodiments, by providing a method and apparatus for determining a patient's brain disease state from brain images, diagnostic errors are minimized and consistent results are provided through an automated analysis process, thereby supporting medical staff to perform more accurate assessment.

Although the embodiments have been described with reference to the accompanying drawings, those skilled in the art will understand that various modifications and changes may be made thereto without departing from the spirit and scope of the invention as defined by the appended claims.

For example, even if the described techniques are performed in a different order from the described method, or if the components of the described system, structure, device, circuit, etc. are combined or combined in a different form from the described method, or replaced or substituted by other components or equivalents, appropriate results may be achieved.

In particular, in the case of describing with reference to the flowchart, although a plurality of steps are configured and the steps are described as being sequentially executed according to the designated order, it is not necessarily limited to the described order.

In other words, it is also applicable as an embodiment to change or delete at least some of the steps described in the flowchart, to add at least one step, and to execute one or more steps in parallel. That is, the steps are not necessarily limited to operating in chronological order, and this should also be included in the embodiments of the present disclosure.

Therefore, other implementations, other embodiments, and equivalents to the claims should also be considered to fall within the scope of the following claims.

What is claimed is:

1. A method of assessing a brain disease, the method comprising:
acquiring a set of brain images of a patient;
partitioning the brain images into a plurality of predefined regions based on anatomical landmarks;
extracting feature information from the plurality of regions, wherein the feature information is indicative of a disease state;
generating, based on a pre-trained brain disease assessment model, a biomarker associated with the disease from the extracted feature information, wherein the biomarker is generated in at least one of the plurality of regions; and
determining a status of the brain disease in the patient based on the biomarker.

2. The method according to claim 1, wherein the plurality of regions includes:
a tight high-convexity region of the cerebrum,
a region encompassing enlarged Sylvian fissures,
a region exhibiting ventriculomegaly, and
an intracranial region.

3. The method according to claim 1, wherein the state of the disease comprises normal pressure hydrocephalus or Alzheimer's disease.

4. The method according to claim 1, wherein the extracting of the feature information from the plurality of regions comprises:
extracting, from each of the plurality of regions, imaging features comprising at least one of volume, shape, signal intensity, and texture.

5. The method according to claim 1, wherein determining the status of the brain disease in the patient comprises:
analyzing a quantitative value of the biomarker; and
comparing the quantitative value to a predetermined reference state, thereby assessing a risk of normal pressure hydrocephalus or Alzheimer's disease.

6. The method according to claim 1, wherein determining the status of the brain disease in the patient comprises:
predicting a presence and progression of the disease based on the biomarker and the feature information.

7. The method according to claim 1, wherein the brain disease assessment model is generated by:
pre-configuring an artificial intelligence model to generate the biomarker associated with the disease in one of a plurality of regions partitioned within training brain images, based on feature information extracted from the training brain images; and
training the artificial intelligence model.

8. The method according to claim 7, further comprising, prior to pre-configuring the artificial intelligence model, performing at least one of:
acquiring a dataset comprising brain images from a plurality of patients as the training brain images;
partitioning the training brain images within the dataset into a plurality of regions; and
extracting feature information for detecting the disease state from the plurality of regions of the training brain images.

9. The method according to claim 7, wherein the brain disease assessment model is trained to:
partition the training brain images into a plurality of regions; or
extract feature information for detecting the state of the disease from the plurality of regions of the training brain images.

10. The method according to claim 8, further comprising:
re-training the brain disease assessment model based on the dataset further comprising brain images of the patient.

11. A brain disease determination apparatus, comprising:
a memory storing a brain image processing model trained to generate a biomarker associated with the disease; and
a processor configured to:
acquire brain images of a patient;
partition the brain images into a plurality of predefined regions based on anatomical criteria;
extract feature information for detecting a state of the disease from the plurality of regions;
generate, based on the brain image processing model, a biomarker associated with the disease from the feature information in at least one of the plurality of regions; and
determine a status of the brain disease in the patient based on the biomarker.

12. The apparatus according to claim 11, wherein the processor is configured to partition the brain images into:
a tight high-convexity region of the cerebrum,
a region encompassing enlarged Sylvian fissures,
a region exhibiting ventriculomegaly, and
an intracranial region.

13. The apparatus according to claim 11, wherein the state of the disease comprises normal pressure hydrocephalus or Alzheimer's disease.

14. The apparatus according to claim 11, wherein the processor is configured to extract, from each of the plurality of regions, imaging features comprising at least one of volume, shape, signal intensity, and texture.

15. The apparatus according to claim 11, wherein the processor is configured to:
analyze a quantitative value of the biomarker; and
compare the quantitative value to a predetermined reference state, thereby assessing a risk of normal pressure hydrocephalus or Alzheimer's disease.

16. The apparatus according to claim 11, wherein the processor is configured to predict a presence and progression of the disease based on the biomarker and the feature information.

17. The apparatus according to claim 11, wherein the brain disease assessment model is generated by:
pre-configuring an artificial intelligence model to generate a biomarker associated with the disease in one of a plurality of regions partitioned within training brain images, based on feature information extracted from training brain images; and
training the artificial intelligence model.

18. The apparatus according to claim 17, wherein, prior to pre-configuring the artificial intelligence model, the processor is configured to perform at least one of:
acquiring a dataset comprising brain images from a plurality of patients as the training brain images;
partitioning the training brain images within the dataset into a plurality of regions; and
extracting feature information for detecting the disease state from the plurality of regions of the training brain images.

19. The apparatus according to claim 17, wherein the brain disease assessment model is trained to:
partition the training brain images into a plurality of regions; or
extract feature information for detecting the state of the disease from the plurality of regions of the training brain images.

20. The apparatus according to claim 18, wherein the processor is configured to re-train the brain disease assessment model based on the dataset further comprising brain images of the patient.
